(19) United States
(12) Patent Application Publication
(10) Pub. No.: US 2020/0407757 A1
(43) Pub. Date: Dec. 31, 2020

(54) METHOD OF PRODUCING ALCOHOL FROM MILK PERMEATE AND COMPOSITIONS THEREOF

(71) Applicant: LIQUOR LABS INCORPORATED, Almonte (CA)

(72) Inventor: Omid McDonald, Almonte (CA)

(21) Appl. No.: 16/837,815

(22) Filed: Apr. 1, 2020

Related U.S. Application Data
(60) Provisional application No. 62/829,111, filed on Apr. 4, 2019.

(30) Foreign Application Priority Data
May 15, 2019 (CA) 3043401

(51) Int. Cl. C12P 7/06; B01D 3/00; C12G 3/02; B01D 3/36; B01D 61/36; C12G 3/04; A23C 9/142

(52) U.S. Cl. CPC C12P 7/06; B01D 3/003; C12G 3/02; A23C 9/1422; B01D 61/362; C12G 3/04; B01D 3/36

(57) ABSTRACT

Provided herein is a method for producing alcohol. The method comprises fermenting milk permeate with yeast to produce a fermented broth comprising the ethanol. The fermented broth is then subjected to distillation to obtain a concentrated ethanol-enriched vapour having at least 50% v/v ethanol. From the ethanol-enriched vapour resulting from the distillation, an ethanol product is produced that is a biofuel or a potable spirit. The disclosure also provides a unique potable spirit composition produced from such process.

### PRIORITY

This U.S. Utility patent application claims priority from U.S. provisional Ser. No. 62/829,111 filed Apr. 4, 2019 and Canadian Patent Application No. 3,043,401 filed May 15, 2019, the contents of which are hereby incorporated by reference in their entirety.

### TECHNICAL FIELD

Provided is a method for producing alcohol from milk permeate.

### BACKGROUND

A by-product of cheese making is a liquid called whey. Whey is a mixture of water, protein (whey and casein), lactose, minerals and vitamins. For every 100 L of milk processed to make cheese about 83 L of whey is produced. Historically, whey was dumped or used as animal feed. The modern dairy industry produces a vast amount of whey and has developed higher value uses for whey. Once such use is to ferment the lactose in whey to produce alcohol for human consumption or for use as fuel. WO 85/01064 describes how the protein in whey is removed with filtration and the lactose in the resulting whey permeate converted to ethanol using a continuous fermentation with Kluyveromyces fragilis yeast.

Ultra-filtration (UF) technology is increasingly being used by the dairy industry to concentrate the proteins in milk to make them cheaper to ship to cheese and yogurt manufacturers. The high protein milk is known as “ultra filtered milk”. UF milk is made by passing milk under pressure through a membrane. The large molecular weight compounds such as proteins and fats are captured by the membrane to make the UF milk, while the liquid fraction, comprising low molecular weight compounds such as water, lactose and minerals, pass through the membrane resulting in a liquid known as milk permeate. Unlike whey, milk permeate contains very low concentrations of protein. Milk permeate has a superior organoleptic profile compared to whey and/or whey permeates as it is derived directly from milk and has fewer processing steps.

Milk permeate is considered a waste stream and it is common practice to simply discard it, despite the fact that it contains lactose and other potentially valuable components. Moreover, the cost of handling and disposing of the waste product is high due to environmental considerations.

Accordingly, there is a need in the art for a method that can utilize milk permeate to produce useful products and avoid harmful impacts on the environment resulting from its disposal. Such a process would also be desirable in that it could potentially spur economic growth in local agricultural communities.

The present invention seeks to address these problems and/or provide a useful alternative to what is described currently in the art.

### SUMMARY

Provided herein is a method for producing alcohol from milk permeate, and ethanol products produced from such method. Advantageously, the use of milk permeate to produce alcohol not only avoids its disposal costs, which can be significant, but can also produce a valuable vendible product from a material that is otherwise considered a waste product.

For example, in one embodiment, the alcohol produced from the milk permeate is further refined to concentrate the alcohol for use as a transportation biofuel. The use of milk permeate as a starting material to make such fuel alcohol has distinct advantages over the use other agricultural feedstock such as corn. Whereas corn can be used for human consumption, milk permeate has little to no nutritional benefit to humans. Growing corn to make biofuel takes up valuable arable land, whereas milk permeate is a waste product of the existing milk processing system. Making biofuels with milk permeate also has distinct advantages over the use of cellulosic material, such as from agricultural waste. To produce ethanol from cellulosic material, the feedstock must first be subjected to an energy and chemically intensive pretreatment. However, such pretreatment liberates inhibitors that drastically reduce the efficiency of enzymatic hydrolysis and the subsequent ethanol fermentation. By contrast, milk permeate is a relatively pure stream of lactose and is generally free of such fermentation inhibitors. Cellulosic material is also subject to seasonal production and suffers from logistical challenges with respect to transporting the material to an ethanol plant. Milk permeate, on the other hand, is a feedstock that is readily available throughout the year, easily transportable to a fermentation facility using existing infrastructure, and is generally free of bacterial contaminants.

A further use of the ethanol resulting from the fermentation of the milk permeate is to produce potable spirits. It has been found by the inventors that potable spirits produced from the milk permeate have unique organoleptic properties that are not present in commercially available spirits produced from conventional starting materials. For example, a vodka product produced from milk permeate has been found to have a sweeter nose than a standard vodka and a smoother finish.

In order to produce the alcohol in a cost-effective manner it is desirable to increase the initial concentration of the sugars to the fermentation. Dilute feed streams result in lower final ethanol concentrations, which dramatically increases the operating costs and carbon footprint associated with the distillation process. Additionally, dilute feed streams increase the capital costs as the equipment used to process the streams increase in size. It is proposed that the milk permeate is concentrated to a range of about 7%-30% w/w with a lactose concentration comprising of approximately 70%-90% w/w of the total solids.

Additionally, while prior fermentation processes described in the literature suggest the use of continuous processes, such processes are not typically economical on a commercial scale. This is due to the susceptibility of continuous processes to microbial contamination. This invention can have a batch or fed-batch fermentation process, thereby allowing for frequent cleaning of the system to avoid contamination issues.

### BRIEF DESCRIPTION OF DRAWINGS

FIG. 1 is a flow diagram depicting a process according to an embodiment of the disclosure. The figure depicts the production of milk permeate via ultrafiltration, with an optional concentration by reverse osmosis step, fermentation of the concentrated milk permeate with yeast and distillation to produce an ethanol stream having at least 50% v/v ethanol.

FIG. 2 is a process configuration showing equipment used to ferment the milk permeate and distill the fermented broth comprising ethanol.

FIG. 3 is a Gas Chromatograph Mass Spectrometer (GC-MS) fingerprint showing the chemical composition of a milk permeate spirit as produced herein.

Like reference symbols in the various drawings indicate like elements.

### DETAILED DESCRIPTION

The milk permeate stream arises from the dairy industry upstream of the alcohol production process and is typically produced by a separation method to isolate fat and/or protein (and optionally other components) from milk. The fats and/or proteins are subsequently made into one or more vendible products, while a waste stream comprising lactose remains. A variety of different separation methods can be used to obtain the fats and/or proteins from the milk. Without being limiting, a common separation method is centrifugal separation to remove the bulk of the fat, followed by filtration using membranes, such as ultrafiltration, to remove the proteins and residual fat. The membrane separation retentate comprises proteins and some fats that are made into commercial products. The permeate stream comprising lactose resulting from the separation is subjected to further processing to produce alcohols.

The lactose concentration of the milk permeate may be optionally further concentrated by any suitable method known by those of skill in the art. In one embodiment, the milk permeate from the separation is further concentrated by reverse osmosis. The permeate subjected to further concentration may be sent to the ethanol plant or such concentration can optionally be carried out at the plant itself. In another embodiment, milk permeate from the protein and/or fat removal steps described above may be sent to the ethanol plant without additional concentration.

As noted, the input stream to fermentation has a total solids concentration in a concentration range of between 7% w/w and 30% w/w, wherein the milk permeate is optionally diluted to achieve said concentration range. In another embodiment, the total solids concentration is between 7% w/w and 22% w/w or between 7% w/w and 20% w/w or between 7% w/w and 18% w/w. In one embodiment, the milk permeate has a total solids concentration of 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% w/w.

As discussed, to achieve the foregoing concentration range of total solids, it may be necessary to dilute the milk permeate that is received by the ethanol plant. The dilution is carried out by the addition of water that may be process water, incoming municipal water, water from a well and/or other water sources. The water need not be pure and can contain a certain level of impurities. It will also be appreciated that the water dilution is an optional step since the milk permeate may already be at a desired concentration when received by the alcohol plant. Without being limiting, if water is added to the incoming milk permeate, the concentration may be adjusted to between 7% w/w and 22% w/w or between 7% w/w and 20% w/w.

The milk permeate is subsequently fed to a fermentation stage that employs yeast. The fermentation converts at least a portion of the lactose in the milk permeate to alcohol. Lactose is a sugar dimer composed of glucose and galactose. The yeast may be capable of fermenting lactose to alcohol either naturally or by genetic manipulation using directed evolution and/or targeted gene modification techniques. Other yeast may be utilized as well that are not capable of directly fermenting lactose to alcohol. In such latter embodiments, prior to fermentation, the lactose is broken down into its component simple sugars, glucose and galactose, by a hydrolysis. The hydrolysis may be carried out by chemical or enzymatic hydrolysis. In one embodiment, chemical hydrolysis is carried out by using an acid, such as sulfuric acid, sulfur dioxide or other acids. Optionally alkali could be used to carry out the hydrolysis. In another embodiment, enzymatic hydrolysis is conducted using an enzyme such as beta-galactosidase.

Examples of suitable yeast include those of the genus Saccharomyces and Kluveromyces. Particular species that may be employed, without limitation, include naturally or genetically modified Saccharomyces cerevisiae and Kluveromyces marxianus.

The fermentation is most advantageously performed at or near the temperature and pH optima of the fermentation microorganism. A typical temperature range for the fermentation of glucose to ethanol is between about 20° C. and about 40° C. The pH of a typical fermentation employing yeast is between about 3 and about 7. The dose of the fermentation microorganism will depend on several factors, such as the activity of the fermentation microorganism, the desired fermentation time, the volume of the reactor and other parameters. It should be appreciated that these parameters may be adjusted as desired by one of skill in the art to achieve optimal fermentation conditions.

The fermentation may be conducted in batch or fed-batch mode. A batch or fed-batch process includes processes in which product is not withdrawn until the end of a run. This is distinguished from a continuous process in which product and reactant are continuously introduced and removed from a reactor. It has been found that batch processes are less prone to contamination. This is a significant advantage since contamination of a continuous system can lead to significantly reduced yields until the entire system can be taken down and cleaned, which is time consuming. By contrast, batch fermentations are more resilient to contamination as they are cleaned frequently and the cleaning systems are incorporated into the design, reducing the severity of impact to contamination.

In one embodiment, the fermentation reactors operated in batch or fed-batch mode are agitated lightly with mechanical agitation. The fermentation microorganisms may be recycled back to the fermenter, or may be sent to distillation without recycle.

The milk permeate fed to fermentation may also be supplemented with additional nutrients required for growth of the fermentation microorganism. For example, yeast extract, specific amino acids, phosphate, nitrogen sources, salts, trace elements and vitamins may be added to the milk permeate to support growth of the microorganism. Ammonia compounds, such as ammonium salts, ammonium hydroxide or ammonia, may also be added to the fermentation media.

The alcohol is subsequently separated from the fermentation broth (in some embodiments referred to as “beer”) by distillation. The fermented broth may comprise between 2% v/v and 15% v/v alcohol. As used herein, the term “distillation” also encompasses steam and vacuum stripping. The fermentation broth or beer may in some embodiments be degassed to remove carbon dioxide and then pumped through one or more distillation columns to separate the alcohol from the other components in the beer. The column(s) in the distillation unit are generally operated in continuous, batch or fed-batch mode. Furthermore, the column(s) may be operated at any desired pressure or vacuum. Heat for the distillation process may be added at one or more points either by direct steam injection or indirectly via heat exchangers. The distillation unit may contain one or more separate beer and rectifying columns, or a distillation column may be employed that comprises an integral enriching or rectification section. As used herein, the term “distillation column” refers to a distillation column, a beer column, a distillation column with a rectification section, a rectification column or a stripper column. When separate beer and rectifying columns are employed, dilute beer is sent to the beer column where it is partially concentrated. From the beer column, the vapour goes to a rectification column for further purification.

An alcohol-enriched vapour, predominantly comprised of the alcohol, is produced during the distillation process as an overhead stream. For biofuel production, the alcohol enriched vapour is further fed to an azeotrope breaking process to remove most of the remainder of water, and subsequently condensed to produce a liquid product typically having at least 99% v/v ethanol. Water removal is required for use of the ethanol in combustion engines since water can contribute to engine corrosion. For spirit production, the alcohol enriched vapour is removed from the distillation system at the appropriate concentration and purity as defined by the spirit type, condensed and then mixed with liquid, such as water or an additive, to produce a spirit product. Processes for making both biofuel and spirits from the condensed ethanol vapour are described in turn below.

In connection with biofuel production, the term “azeotropic breaking process” is meant to encompass any process for breaking the azeotrope of the ethanol-enriched vapour to produce the concentrated biofuel product. This includes, but is not limited to, feeding the ethanol-enriched vapour to molecular sieves. Other azeotropic breaking processes that are encompassed by this definition include pervaporation and the addition of benzene or cyclohexane to a distillation column. After breaking the azeotrope to obtain the concentrated ethanol solution, the vapour is typically condensed to product ethanol and then denatured.

Turning now to spirit production, the concentrated ethanol product having at least 50% v/v alcohol is typically diluted to prepare a spirit. As set out in more detail below, this generally involves diluting the concentrated ethanol to below about 45% v/v ethanol with water or a liquid additive.

The production of spirits from the concentrated and condensed ethanol solution varies depending on the particular type of spirit being produced. As used herein, the term “spirit” refers to a potable ethanol composition with an ethanol concentration higher than 20% v/v. In further embodiments, the ethanol concentration is higher than 22% v/v, 24% v/v, 26% v/v, 28% v/v or 30% v/v.

It will be appreciated that the particular name assigned to a category of spirit as used herein, such as vodka, gin, whiskey, brandy, or the like, includes not only any spirit that can be labelled and/or marketed in any country or jurisdiction as such, but includes also a similar product that is marketed under a similar or different name. As those of skill in the art will appreciate, spirits in any particular country or jurisdiction often must meet certain legal definitions regarding the starting material for alcohol production and/or the method of production. To illustrate, a vodka produced using the starting materials as set forth herein, may be produced in a similar manner and have a similar taste and/or chemical composition as a vodka meeting the requirements of regulations, yet may not be considered “vodka” due to differences in starting material, manufacturing process and/or composition of end product, optionally among other requirements, as defined by regulators. Thus, the particular name that can be assigned to a particular spirit, as used herein, is not limited to any particular definition applied by regulators of a particular country or jurisdiction. To illustrate, a vodka product marketed and labelled as “VODKOW™” may or may not meet the definition of vodka in a particular country or jurisdiction, but nonetheless will be considered “vodka” for the purposes herein due to similarities in taste to traditional vodka.

For spirit production, the concentrated alcohol from distillation, which may have an alcohol concentration of around 40% to 97.2% v/v, may be mixed with a liquid, typically water or other liquid solutions, with the optional addition of one or more flavoured additives. The addition of liquid serves to reduce the alcohol concentration to between 20% v/v and 60% v/v, more typically between 30% v/v and 50% v/v or between 35% v/v and 45% v/v.

According to another embodiment, the concentrated alcohol is used to make a liquor product comprising one or more flavour and/or colour additives. Non-limiting examples of such products include a liqueur, cordial or schnapps. In one embodiment, the concentration of alcohol in the liqueur is greater than 13% v/v. The liqueur can be a cream liqueur, although other liqueurs are included within the scope of the disclosure. In one example, the liqueur comprises the concentrated alcohol, cream and maple syrup.

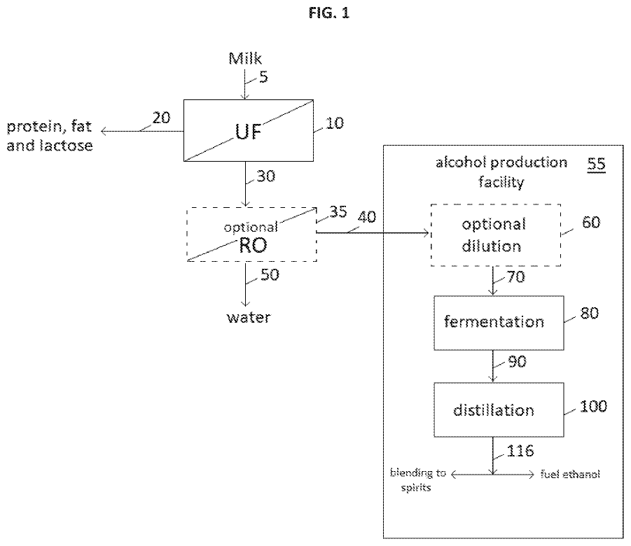

Turning now to FIG. 1, there is provided a block flow diagram that depicts a non-limiting example of how the method to produce an alcohol product at a concentration of at least 50% w/w from milk permeate can be carried out. According to the method, skim milk 5 having 5% w/w total solids and comprising lactose, protein and residual fat is subjected to ultrafiltration 10. The ultrafiltration 10 employs a membrane of suitable pore size so as to produce a retentate stream 20 comprising the protein, residual fat and lactose and a milk permeate stream 30 comprising 5% w/w lactose and other components. The retentate stream 20 is used to produce ultra-filtered milk, yogurt or cheese due to its high protein content. Rather than being disposed of, the milk permeate stream 30 is subjected to a fermentation to produce the alcohol, as described below. Prior to fermentation, the milk permeate stream 30 is optionally concentrated by reverse osmosis 35 to produce concentrated milk permeate 40 comprising 22% w/w total solids and a water stream 50 that is disposed of.

The concentrated milk permeate 40 is subsequently sent to an alcohol production facility 55 where permeate 40 is optionally diluted in a dilution stage 60 to achieve a total solids content of 7% w/w to 20% w/w in the fermentation input stream 70. As discussed previously, the concentration of total solids (w/w) is maintained within a range that minimizes capital costs while keeping within the fermentative capabilities of the yeast to make the method economically feasible. The fermentation input stream 70 is sent to a fermentation 80 that is part of alcohol production facility 55. The fermentation 80 is carried out in batch or fed-batch for ease of cleaning process equipment, which further contributes to the economic feasibility of the method, as described previously. The fermentation 80 results in a fermented broth 90 having about 5% v/v alcohol. The fermented broth 90 is subsequent subjected to distillation 100. The distillation 100 produces a concentrated ethanol stream 116 having at least 50% v/v ethanol.

If a biofuel is the desired product, the concentrated ethanol stream 116 can be subjected to a further water removal step utilizing molecular sieves, pervaporation or other known methods to break the azeotrope of 97.2% v/v ethanol. If a potable spirit is the desired product, additives can be mixed with the concentrated ethanol stream 116 to produce a final vendible spirit product rather than concentrating the stream 116 beyond the azeotropic breaking point.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

The invention will be further described with reference to the examples described herein; however, it is to be understood that the invention is not limited to such examples.

### EXAMPLES

### Example 1: Process Configuration for Fermenting Milk Permeate to Ethanol and

Concentration

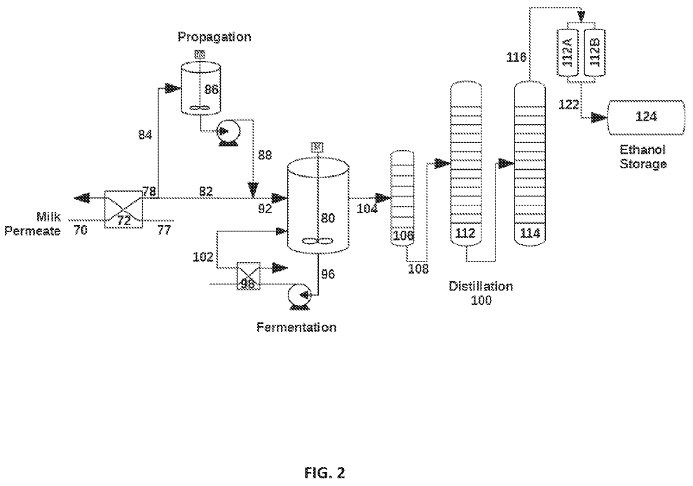

FIG. 2 depicts a specific configuration of equipment for fermenting the milk permeate input stream 70 to ethanol, followed by concentration by distillation to yield a concentrated ethanol stream 116 having at least 90% (v/v) ethanol, more typically at least 95% (v/v) ethanol. It will be appreciated that this example is illustrative only and various other embodiments and alternatives are contemplated herein.

The milk permeate input stream 70 is adjusted to the fermentation temperature by heat exchanger 72 in which the input stream 70 is heated by exchange against a warm process stream 77. The heated stream 78 is split into a main stream 82 and a slip stream 84. The main stream 82 is sent to fermentation 80 and the slip stream 84 is sent to a yeast propagation unit 86. Yeast-containing stream 88 from the yeast propagation unit 86 is pumped via a pump to the main stream 82 and combined with same to produce a combined stream 92 fed to the fermentation 80, which utilizes a fed-batch fermentation tank. A stream 96 is withdrawn from fermentation 80 via a pump and heat exchanged in a heat exchanger 98 employed to cool stream 96. An outlet stream 102 from the heat exchanger 98 is re-introduced to fermentation 80 to maintain a constant fermentation temperature. A fermented stream 104 (often referred to as “beer”) having about 5% v/v ethanol is fed to distillation 100.

In the distillation 100, the fermented stream 104 is fed to a de-gas column 106, in which carbon dioxide is removed from fermented stream 104. A de-gassed stream 108 is sent to a beer column 112 and subsequently to a rectifier column 114. An overhead ethanol-enriched vapour stream 116 from the rectifier column 114 having an ethanol concentration of about 95% v/v may be fed to molecular sieves 112A and 112B to remove additional water to produce a de-watered ethanol stream 122 having greater than 98% v/v ethanol for biofuel production. The de-watered ethanol stream 122 is stored in an ethanol storage tank 124. Alternatively, the 95% v/v overhead stream 116 is condensed and blended with additives to produce potable spirits, such as a vodka.

### Example 2: Chemical Composition of Milk Permeate Spirit

Samples of a milk permeate spirit produced as described herein (Vodkow™), and two commercially available vodka spirits were analyzed by gas chromatography mass spectrometry (GC/MS) to determine their chemical compositions.

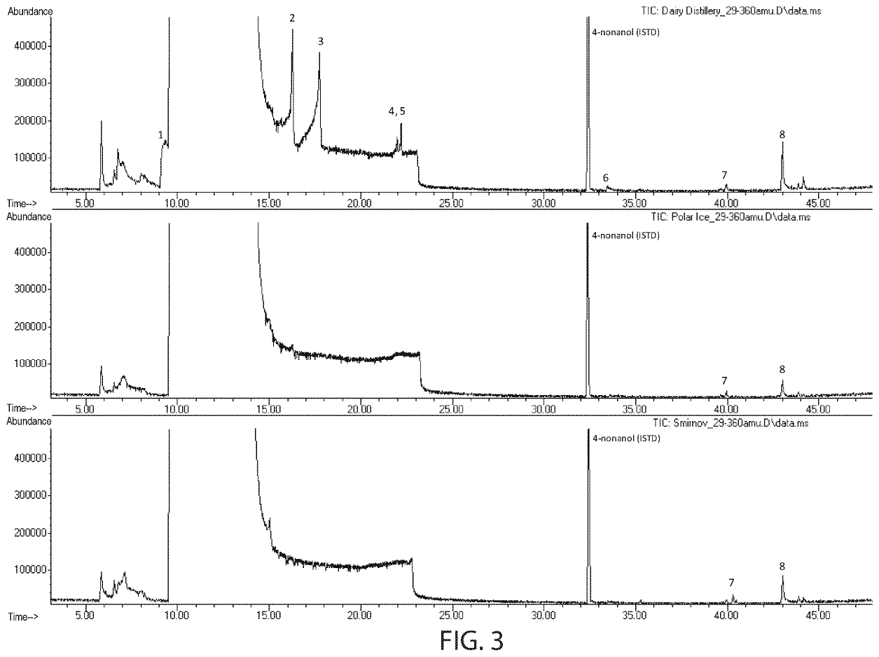

FIG. 3 is a Gas Chromatograph Mass Spectrometer (GC-MS) fingerprint showing a full scan of the three spirits analyzed: Vodkow™ (top), Polar Ice™ (middle) and Smirnoff™ (bottom) over time on the X-axis. The first six peaks seen in Vodkow™ that are not found in the other spirits are, in order of appearance: 1-propanol, isobutyl alcohol, 2-methylbutan 1-ol, isoamyl acetoacetate and acetic acid.

Table 1 below outlines the components that were found and searched for as part of the GC-MS fingerprinting work.

TABLE 1
Major volatiles measured in micrograms per
millilitre (ppm) for three different vodka
brands: VODKOW ™, POLAR ICE ™ and SMIRNOFF ™.

| | VODKOW ™ | POLAR ICE ™ | SMIRNOFF ™ |
|---|---|---|---|
| Compound | μg/mL | μg/mL | μg/mL |
| Methanol | N.D. | N.D. | N.D. |
| 1-Propanol | 60.14 | N.D. | N.D. |
| Isobutyl alcohol | 48.63 | N.D. | N.D. |
| 1 Butanol | N.D. | N.D. | N.D. |
| Isoamyl alcohol | 44.37 | N.D. | N.D. |
| Isopentyl alcohol | 52.82 | N.D. | N.D. |
| Acetic acid | Below detection | N.D. | N.D. |
| | limit | | |

The data collectively shows that VODKOW™ has a unique chemical composition relative to commercially available vodkas.

The other compounds that were tested, but were not detected, are acetone, ethyl carbamate, 2-butanol, ethyl butyrate, butanoic acid, ethyl isovalerate, isoamyl acetate, ethyl valerate, heptaldehyde, ethyl caproate, isoprenol, 2-heptanol, 3-methyl-1-pentanol, ethyl heptanoate, ethyl lactate, 1-hexanol, ethyl caprylate, 1-heptanol, ethyl acetate, furfural, ethyl pelargonate, linalool, benzaldehyde, 1-octanol, ethyl caprate, ethyl laurate, isovaleric acid, 2-methylbutyric acid, diethyl succinate, ethyl 9-decenoate, a-terpineol, ethyl undecanoate, geraniol, caproic acid, ethyl myristate, caprylic acid, nonaoic acid, ethyl palmitate, geranic acid, ethyl palmitate, ethyl stearate and dodecanoic acid.

It should be appreciated that the foregoing is simply a description of an example and that other embodiments and variations not described herein fall within the scope of the invention as defined by the claims appended hereto.

What is claimed is:

1. A method for producing alcohol comprising:
(i) providing a milk permeate having a total solids concentration in a concentration range of between about 7% w/w and about 30% w/w of which about 70% to 90% is lactose, wherein the milk permeate is optionally diluted to achieve said concentration range;
(ii) fermenting the milk permeate of step (i) to produce a fermented broth comprising the alcohol;
(iii) subjecting the fermented broth to distillation to obtain a concentrated alcohol-enriched vapour of at least about 50% v/v ethanol; and
(iv) from the alcohol-enriched vapour resulting from the distillation, producing an alcohol product that is a biofuel or a potable spirit,
wherein steps (ii) and (iii) are carried out in batch or fed-batch.

2. The method of claim 1, wherein the product of step (iv) is a potable spirit and is selected from vodka, whiskey, whisky, gin, brandy or a liqueur.

3. The method of claim 1, wherein the potable spirit is produced by condensing the alcohol-enriched vapour to produce a condensed solution and combining water or an additive with the condensed solution to produce the potable spirit.

4. The method of claim 3, wherein the potable spirit is vodka.

5. The method of claim 1, wherein the product is the biofuel.

6. The method of claim 5, wherein the product is a biofuel with a carbon intensity less than about 30% of gasoline as measured by a life cycle analysis model such as GREET or GHGenius.

7. The method of claim 5, wherein the biofuel is produced from the concentrated solution resulting from distillation by further removal of water from the concentrated solution by an azeotropic breaking process selected from concentration by molecular sieves or pervaporation.

8. The method of claim 1, wherein the milk permeate of step (i) has a total solids concentration of between about 7% w/w to about 22% w/w.

9. The method of claim 1, wherein the milk permeate of step (i) is obtained by subjecting an incoming milk permeate solution having a concentration of about 15% to 22% w/w total solids to the dilution, and wherein said dilution comprises adding water to the incoming milk permeate to achieve a milk permeate solution having a total solids concentration of between about 7% w/w and 18% w/w that is fed to the step of fermenting.

10. The method of claim 1, wherein the fermenting does not comprise a membrane separation.

11. A method for producing a potable spirit comprising:
(i) providing a milk permeate and optionally diluting said permeate;
(ii) fermenting the milk permeate of step (i) with yeast to produce a fermented broth comprising the alcohol;
(iii) subjecting the fermented broth to distillation to obtain a concentrated alcohol having at least about 50% v/v ethanol; and
(iv) from the concentrated alcohol resulting from the distillation, producing the potable spirit by addition of a liquid to the concentrated solution, wherein the potable spirit has greater than about 20% v/v ethanol.

12. The method of claim 11, wherein the potable spirit is produced by combining an additive with the concentrated alcohol resulting from the distillation.

13. The method of claim 11, wherein the potable spirit is vodka, whiskey, whisky, gin, brandy or a liqueur.

14. The method of claim 11, wherein the milk permeate of step (i) has a total solids concentration of between 7% w/w and 30% w/w.

15. The method of claim 11, wherein steps (ii) and (iii) are carried out in batch or fed-batch.

16. The method of claim 11, wherein the fermenting does not comprise a membrane separation.

17. A potable spirit produced by the method of claim 1.

18. A potable spirit produced by the method of claim 11.

19. The potable spirit of claim 17, wherein the spirit is vodka.

20. A potable spirit or a biofuel comprising: ethanol, water and at least one compound selected from 1-propanol, isobutyl alcohol, isoamyl alcohol and isopentyl alcohol.
